(19) United States
(12) Patent Application Publication
(10) Pub. No.: US 2009/0088576 A1
(43) Pub. Date: Apr. 2, 2009

(54) Process for the Stereoselective Preparation of Alcohols From Alpha, Beta-Insaturated Compounds

(76) Inventors: Peter Herold, Allschwil (CH); Stefan Stutz, Allschwil (CH); Robert Mah, Allschwil (CH); Aleksandar Stojanovic, Allschwil (CH); Isabelle Lyothier, Allschwil (CH); Dirk Behnke, Allschwil (CH); Felix Spindler, Starrkirch-Wil (CH); Erhard Bappert, Basel (CH)

Correspondence Address: WENDEROTH, LIND & PONACK, L.L.P., 2033 K STREET N. W., SUITE 800, WASHINGTON, DC, 20006-1021, US

(21) Appl. No.: 12/223,338

(22) PCT Filed: Jan. 29, 2007
(86) PCT No.: PCT/EP2007/050816

§ 371 Date: Jul. 29, 2008

(30) Foreign Application Priority Data
Jan. 30, 2006 (CH) 00149/06
Dec. 14, 2006 (CH) 02029/06

(51) Int. Cl. C07D 231/56

(52) U.S. Cl. 548/362.5

(57) ABSTRACT

A process for the synthesis of an alcohol of formula (I), using as intermediate a compound of formula (V).

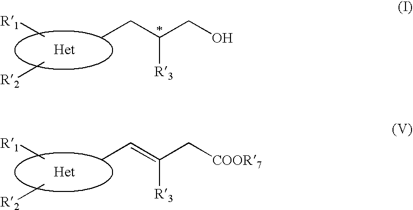

The invention relates to a stereoselective process for the preparation of (R or S)-2-alkyl-3-heterocyclyl-1-propanols and of novel intermediates which are obtained in the process stages.

WO 2005/090305 A1 discloses δ-amino-γ-hydroxy-ω-(heterocyclyl)alkanecarboxamides which exhibit renin-inhibiting properties and can be used as antihypertensive agent in pharmaceutical compositions. The preparation processes disclosed therein, which proceed via a coupling of a heterocyclyl-metal entity to an aldehyde as key step, are unsuitable for an industrial process, in particular in view of the unsatisfactory yields in some cases.

In a new process, the starting material is 2,7-dialkyl-8-heterocyclyl-4-octenoylamides, the double bond of which is simultaneously halogenated in the 5 position and hydroxylated with lactonization in the 4 position, then the halogen is replaced with azide, the lactone is amidated and the azide is then converted to the amine group. The desired alkane-carboximides are obtained in this new process in appreciably higher overall yields. The halolactonization, the azidation and the azide reduction are carried out following the process described by P. Herold in the Journal of Organic Chemistry, Vol. 54 (1989), pages 1178-1185.

The 2,7-dialkyl-8-heterocyclyl-4-octenoylamides can, for example, correspond to the formula A,

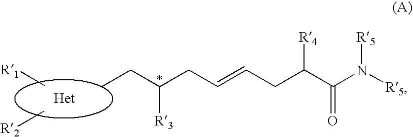

in which Het represents an unsaturated bicyclic heterocyclyl joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, R′₃ represents C₁-C₈-alkyl, R′₄ is C₁-C₈-alkyl, R′₅ represents C₁-C₈-alkyl or C₁-C₈-alkoxy, R′₆ represents C₁-C₈-alkyl or R′₅ and R′₆ together are tetramethylene, pentamethylene, 3-oxa-1,5-pentylene or —CH₂CH₂O—C(O)— optionally substituted by C₁-C₄-alkyl, phenyl or benzyl, and in which the carbon atom to which the R′₃ radical is bonded exhibits either the (R) or (S) configuration, the (R) configuration being preferred.

The compounds of the formula A can be obtained by reacting a compound of the formula B

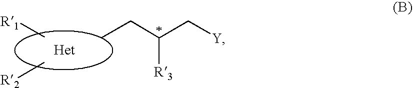

with a compound of the formula C,

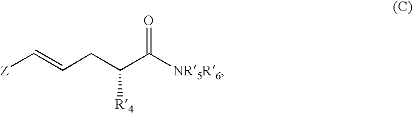

in which Het, R′₁ to R′₄, R′₅ and R′₆ have the meanings given above, Y represents Cl, Br or I and Z represents Cl, Br or I and in which the carbon atom to which the R′₃ radical is bonded exhibits either the (R) or (S) configuration, the (R) configuration being preferred, in the presence of an alkali metal or alkaline earth metal. Y and Z preferably represent Br or Cl and particularly preferably Cl.

The compounds of the formula C can be prepared by amidation of the corresponding carboxylates, carboxamides or carboxylic acid halides. The formation of carboxamides from carboxylates and amines in the presence of trialkylaluminium or dialkylaluminium halide, for example with trimethylaluminium or dimethylaluminium chloride, is described by S. M. Weinreb in Organic Syntheses, 59, pages 49-53 (1980). The carboxylates can be obtained by the reaction of trans-1,3-dihalopropene (for example trans-1,3-dichloropropene) with appropriate carboxylates in the presence of strong bases, for example alkali metal amides.

The stereoselective preparation of compounds of the formula B is not yet known. It has now been found, surprisingly, that 2-alkyl-3-heterocyclyl-1-propanols (compounds of the formula B with Y in the OH meaning; subsequently described as compound of the formula I) can be prepared stereospecifically in high yields in only four or five process stages; if, analogously to the process disclosed in WO 02/02487 A1, suitably substituted, unsaturated, heterocyclylaldehydes are condensed with carboxylates to give 2-alkyl-3-hydroxy-3-(heterocyclyl)carboxylates, the diastereomeric products are obtained in high yields. It has been found, surprisingly, that the diastereomers obtained, in the case of the 2-alkyl-3-hydroxy-3-(heterocyclyl)carboxylates, are advantageously not separated since, after converting the hydroxyl group to a leaving group, followed by base-induced elimination, (E)-3-heterocyclyl-2-alkylacrylates are formed with high stereoselectivity. The (E)-3-heterocyclyl-2-alkylacrylates are an important intermediate of the process. Starting from these (E)-3-heterocyclyl-2-alkylacrylates, the 2-alkyl-3-heterocyclyl-1-propanols can be obtained by three process variants:

1) From the crude 3-heterocyclyl-2-alkylacrylic acids, after saponification and crystallization, exclusively (E)-3-heterocyclyl-2-alkylacrylic acids are obtained in high yields. The (E)-3-heterocyclyl-2-alkylacrylic acids can, in the presence of specific catalysts, be hydrogenated to give virtually enantiomerically pure 2-alkyl-3-heterocyclyl-1-propionic acids which, by reduction, can be converted to 2-alkyl-3-heterocyclyl-1-propanols of the formula I.

2) From the crude 3-heterocyclyl-2-alkylacrylic acids, after saponification and crystallization, exclusively (E)-3-heterocyclyl-2-alkylacrylic acids are obtained in high yields. The (E)-3-heterocyclyl-2-alkylacrylic acids can be reduced to give allyl alcohols; the allyl alcohols obtained can in turn be hydrogenated in the presence of specific catalysts to give virtually enantiomerically pure 2-alkyl-3-heterocyclyl-1-propanols of the formula I.

3) The (E)-3-heterocyclyl-2-alkylacrylates can be reduced to give allyl alcohols. The allyl alcohols obtained can in turn be hydrogenated in the presence of specific catalysts to give virtually enantiomerically pure 2-alkyl-3-heterocyclyl-1-propanols of the formula I.

In the process variants 1) and 2), all process steps up to the (E)-3-heterocyclyl-2-alkylacrylic acids are advantageously carried out without purification of the intermediates, which represents a considerable advantage for the preparation on an industrial scale (e.g., cost saving). The process variant 3) is shorter by one process stage, which is likewise advantageous for the preparation on an industrial scale.

The 2-alkyl-3-heterocyclyl-1-propanols of the formula I given below obtained in this way can then be converted by halogenation in a way known per se, for example according to the process described by J. Maibaum in Tetrahedron Letters, Vol. 41 (2000), pages 10085-10089, to the compounds of the formula B.

A subject-matter of the invention is a process for the preparation of compounds of the formula I,

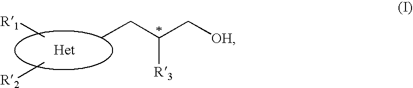

in which Het represents an unsaturated bicyclic heterocyclyl joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, and R′₃ represents C₁-C₈-alkyl, and in which the carbon atom to which the R′₃ radical is bonded exhibits either the (R) or (S) configuration, the (R) configuration being preferred, characterized in that

a) a compound of the formula II

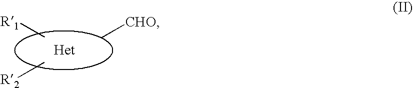

in which Het, R′₁ and R′₂ have the meanings given above, is reacted with a compound of the formula II,

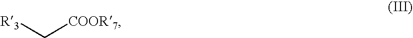

in which R′₃ has the meaning given above, to give a diastereomeric mixture of the formula IV,

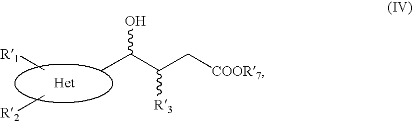

in which R′₇ is C₁-C₁₂-alkyl, C₃-C₈-cycloalkyl, phenyl or benzyl,

b) the OH group of the diastereomeric mixture of the formula IV is converted to a leaving group and the leaving group is eliminated in the presence of a strong base to give an acrylate of the formula V,

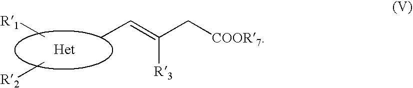

### Process Variant 1

This process variant is characterized in that

1c) the acrylate of the formula V is converted by saponification to give a compound of the formula VI,

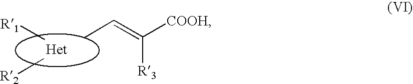

1d) the acid of the formula VI is hydrogenated in the presence of hydrogen and catalytic amounts of a metal complex as asymmetric hydrogenation catalyst, which comprises metals from the group consisting of ruthenium, rhodium and iridium to which chiral bidentate ligands are bonded, to give a compound of the formula VIII,

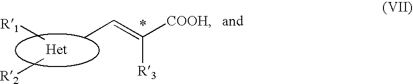

1e) the acid of the formula VII is reduced to give a compound of the formula I.

### Process Variant 2

This process variant is characterized in that

2c) the acrylate of the formula V is converted by saponification to give a compound of the formula VI,

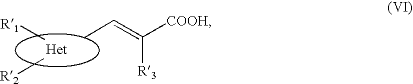

2d) the acid of the formula VI is reduced to give a compound of the formula VIII

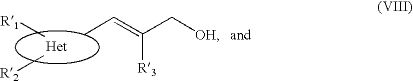

2e) the alcohol of the formula VIII is hydrogenated in the presence of hydrogen and catalytic amounts of a metal complex as asymmetric hydrogenation catalyst, which comprises metals from the group consisting of ruthenium, rhodium and iridium to which chiral bidentate ligands are bonded, to give a compound of the formula I.

### Process Variant 3

This process variant is characterized in that

3c) the acrylate of the formula V is reduced to give a compound of the formula VIII

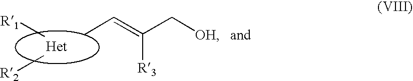

3d) the alcohol of the formula VIII is hydrogenated in the presence of hydrogen and catalytic amounts of a metal complex as asymmetric hydrogenation catalyst, which comprises metals from the group consisting of ruthenium, rhodium and iridium to which chiral bidentate ligands are bonded, to give a compound of the formula I.

R′₁ and R′₂ can, as C₁-C₈-alkyl, be linear or branched and preferably comprise 1 to 4 carbon atoms. Examples are methyl, ethyl, n-propyl, isopropyl, n-butyl, isobutyl, t-butyl, pentyl and hexyl.

R′₁ and R′₂ can, as polyhalo-C₁-C₈-alkyl, be linear or branched and preferably comprise 1 to 4, particularly preferably 1 or 2, carbon atoms. Examples are fluoromethyl, difluoromethyl, trifluoromethyl, chloromethyl, dichloromethyl, trichloromethyl, 2-chloroethyl and 2,2,2-trifluoroethyl.

R′₁ and R′₂ can, as polyhalo-C₁-C₈-alkoxy, be linear or branched and preferably comprise 1 to 4, particularly preferably 1 or 2, carbon atoms. Examples are fluoromethoxy, difluoromethoxy, trifluoromethoxy, chloromethoxy, dichloromethoxy, trichloromethoxy, 2-chloroethoxy and 2,2,2-trifluoroethoxy.

R′₁ and R′₂ can, as halogen, inclusive of halo in polyhalo-C₁-C₈-alkyl and polyhalo-C₁-C₈-alkoxy, represent F, Cl or Br, F and Cl being preferred.

R′₁, R′₂ and R′₅ can, as C₁-C₈-alkoxy, be linear or branched and preferably comprise 1 to 4 carbon atoms. Examples are methoxy, ethoxy, n-propoxy, isopropoxy, n-butoxy, isobutoxy and t-butoxy, pentoxy and hexoxy.

R′₁ and R′₂ can, as C₁-C₈-alkoxy-C₁-C₈-alkyl, be linear or branched. The alkoxy group preferably comprises 1 to 4 and in particular 1 or 2 carbon atoms and the alkyl group preferably comprises 1 to 4 carbon atoms. Examples are methoxymethyl, 1-methoxyeth-2-yl, 1-methoxyprop-3-yl, 1-methoxybut-4-yl, methoxypentyl, methoxyhexyl, ethoxymethyl, 1-ethoxyeth-2-yl, 1-ethoxyprop-3-yl, 1-ethoxybut-4-yl, ethoxypentyl, ethoxyhexyl, propoxymethyl, butoxymethyl, 1-propoxyeth-2-yl and 1-butoxyeth-2-yl.

R′₁ and R′₂ can, as C₁-C₈-alkoxy-C₁-C₈-alkoxy, be linear or branched. One alkoxy group preferably comprises 1 to 4 and especially 1 or 2 carbon atoms and the other alkoxy group preferably comprises 1 to 4 carbon atoms. Examples are methoxymethoxy, 2-methoxyethoxy, 3-methoxypropoxy, 4-methoxybutoxy, methoxypentoxy, methoxyhexoxy, ethoxymethoxy, 2-ethoxyethoxy, 3-ethoxypropoxy, 4-ethoxybutoxy, ethoxypentoxy, ethoxyhexoxy, propoxymethoxy, butoxymethoxy, 2-propoxyethoxy and 2-butoxyethoxy.

In a preferred embodiment, R′₁ represents methoxy- or ethoxy-C₁-C₄-alkyl and R′₂ preferably represents methyl, ethyl, methoxy or ethoxy. Very particular preference is given to compounds of the formula I in which R′₁ represents 3-methoxypropyl or 4-methoxybutyl and R′₂ represents methyl or methoxy.

R′₃, R′₄, R′₅ and R′₆ can, as C₁-C₈-alkyl, be linear or branched and preferably comprise 1 to 4 carbon atoms. Examples are methyl, ethyl, n-propyl, isopropyl, n-butyl, isobutyl, t-butyl, pentyl and hexyl. In a preferred embodiment, in the compounds of the formula I, R′₃ represents isopropyl and the carbon atom to which the R′₃ radical is bonded exhibits the (R) configuration.

Het can, as unsaturated bicyclic heterocyclyl joined via a carbon atom to the residual molecule, comprise unsaturated bicyclic heterocyclic radicals with 1 to 4 nitrogen atoms and/or 1 or 2 sulphur or oxygen atoms, radicals with one or 2 nitrogen atoms being preferred. Preferred bicycles consist in each case of 5- and/or 6-membered rings. Examples for Het are benzothiazolyl, quinazolinyl, quinolyl, quinoxalinyl, isoquinolyl, benzo[b]thienyl, isobenzofuranyl, benzimidazolyl, indolyl, dihydrobenzofuranyl, tetrahydroquinoxalinyl, 3,4-dihydro-2H-benzo[1,4]oxazinyl, 1H-pyrrolizinyl, phthalazinyl, dihydro-2H-benzo[1,4]thiazinyl, 1H-pyrrolo[2,3-b]pyridyl, imidazo[1,5-a]pyridyl, benzoxazolyl, 2,3-dihydroindolyl, indazolyl or benzofuranyl. Het particularly preferably represents 1H-indol-6-yl or 1H-indazol-6-yl.

Particularly preferred are compounds of the formula I in which Het substituted with R′₁ and R′₂ represents 1-(3-methoxypropyl)-3-methyl-1H-indol-6-yl, 3-(3-methoxypropyl)-1-methyl-imidazo[1,5-a]pyridin-6-yl or 1-(3-methoxypropyl)-3-methyl-1H-indazol-6-yl and R′₃ represents isopropyl.

R′₇ can, as C₃-C₈-cycloalkyl, represent cyclopropyl, cyclobutyl, cyclopentyl, cyclohexyl, cycloheptyl or cyclooctyl.

R′₇ preferably represents C₁-C₆-alkyl and particularly preferably C₁-C₄-alkyl; some examples are methyl, ethyl, n-propyl and n-butyl.

The starting compounds of the formulae II and III used in the process stage a) are known or can be prepared analogously to known processes. Compounds of the formula II are prepared in a way known per se from the unsaturated bicyclic heterocyclyl bromides disclosed in WO 2005/090305 A1 via halogen/metal exchange and subsequent reaction with N,N-dimethylformamide. The reaction of the process stage a) is advantageously carried out at low temperatures, for example from 40 to 0° C., in the presence of at least equivalent amounts of a strong base. The reaction is furthermore advisably carried out in a solvent, ethers, such as, for example, diethyl ether, tetrahydrofuran and dioxane, being particularly suitable. Suitable strong bases are in particular alkali metal alkoxides and alkali metal secondary amides, for example lithium diisopropylamide.

The mixture of the two diastereomers of the formula IV is obtained in virtually quantitative yield. The diastereomer mixture is advantageously used without purification in the next process stage.

The conversion of the OH group to a leaving group in the process stage b) is known per se. Reaction with carboxylic acids or sulphonic acids, or the acid chlorides or anhydrides thereof, (acylation) is particularly suitable. Some examples of carboxylic or sulphonic acids are formic acid, acetic acid, propionic acid, benzoic acid, benzenesulphonic acid, toluenesulphonic acid, methylsulphonic acid and trifluoromethylsulphonic acid. The use of acetic anhydride in the presence of catalytic amounts of 4-dimethylaminopyridine has proven to be particularly worthwhile. The elimination is advisably carried out in the presence of strong bases, alkali metal alkoxides, such as potassium tert-butoxide, being particularly suitable. The presence of solvents, such as ethers, is advisable. The reaction is advantageously carried out at low temperatures, for example from 0° C. to 40° C. The elimination reaction is advantageously carried out directly in the reaction mixture of the process stage a). The elimination surprisingly results selectively in the desired E isomers of the acrylates of the formula V.

The saponification of the acrylates of the formula V in the process stages 1c) and 2c) is advantageously carried out directly, after reaching completion of the elimination (process stage b)) and after concentrating the solvent, by addition of, for example, potassium hydroxide solution and stirring at temperatures between 80° C. and 100° C. The acids of the formula VI obtained are highly crystalline and can accordingly be isolated in a simple way without large losses by means of extraction and crystallization. The yields are greater than 60%. Surprisingly, the desired E isomers are exclusively obtained.

Asymmetric hydrogenations analogously to the process stage 1d) of α,β-unsaturated carboxylic acids of the formula VI and to the process stages 2e) and 3d) of α,β-unsaturated alcohols of the formula VIII with homogeneous asymmetric hydrogenation catalysts are known per se and are described, for example, by J. M. Brown in E. Jacobsen, A. Pfaltz and H. Yamamoto (Eds.), Comprehensive Asymmetric Catalysis, I to III, Springer Verlag, 1999, pages 121-182, and by X. Zhang in Chemical Reviews, Vol. 103 (2003), pages 3029-3069. Ruthenium, rhodium and iridium catalysts are particularly effective.

Asymmetric hydrogenations of α,β-unsaturated carboxylic acids of the formula VI can generally preferably be carried out using ruthenium or rhodium catalysts, such as described, for example, by J. M. Brown in E. Jacobsen, A. Pfaltz and H. Yamamoto (Eds.), Comprehensive Asymmetric Catalysis, I to III, Springer Verlag, 1999, pages 163-166, by W. Weissensteiner and F. Spindler in Advanced Synthesis and Catalysis, Vol. 345 (2003), pages 160-164, and by T. Yamagishi in the Journal of the Chemical Society, Perkin Transactions 1, (1997), pages 1869-1873.

Use is frequently made, as ligands for rhodium and ruthenium, of chiral ditertiary bisphosphines. Such chiral ditertiary bisphosphines are described, for example, by X. Zhang in Chemical Reviews, Vol. 103 (2003), pages 3029-3069.

Ligands with a ferrocenyl backbone are generally particularly suitable for the asymmetric hydrogenation of α,β-unsaturated carboxylic acids. Examples are described by F. Spindler in Tetrahedron: Asymmetry, Vol. 15 (2004), pages 2299-2306. Examples are ligands of the Walphos, Josiphos, Mandyphos and Taniaphos families. Ligands of these families are described, for example, by X. Zhang in Chemical Reviews, Vol. 103 (2003), pages 3029-3069, and also by P. Knochel in Chemistry, a European Journal, Vol. 8 (2002), pages 843-852, by H.-U. Blaser in Topics in Catalysis, Vol. 19 (2002), pages 3-16, and by F. Spindler in Tetrahedron: Asymmetry, Vol. 15 (2004), pages 2299-2306.

It has now surprisingly been found that rhodium metal complexes, ligands of which belong to the families of chiral ditertiary bisphosphines with a ferrocenyl backbone, are particularly suitable for the asymmetric hydrogenation of α,β-unsaturated carboxylic acids of the formula VI.

It is possible, with the abovementioned ferrocenyl ligand families in the metal complexes of the formulae IX and IXa described below, to obtain high enantiomeric purities, which represents a considerable advantage (for example, cost saving) for the preparation on an industrial scale.

[LMYZ] (IX),

[LMY]⁺E⁻ (IXa),

in which
M represents rhodium;
Y is two olefins or a diene;
Z represents Cl, Br or I;
E⁻ represents the anion of an oxo acid or complex acid; and
L is a chiral ligand from the group consisting of ditertiary bisphosphines.

When Y has the meaning olefin, C₂-C₁₂-olefins, preferably C₂-C₆-olefins and particularly preferably C₂-C₄-Olefins may be concerned. Examples are propene, butene and in particular ethylene. The diene can comprise 5 to 12 and preferably 5 to 8 carbon atoms and open-chain, cyclic or polycyclic dienes may be concerned. The two olefin groups of the diene are preferably connected by one or two CH₂ groups. Examples are 1,3-pentadiene, cyclopentadiene, 1,5-hexadiene, 1,4-cyclohexadiene, 1,4- or 1,5-heptadiene, 1,4- or 1,5-cycloheptadiene, 1,4- or 1,5-octadiene, 1,4- or 1,5-cyclooctadiene and norbornadiene. Preferably, Y represents two ethylenes or 1,5-hexadiene, 1,5-cyclooctadiene or norbornadiene.

In formula IX, Z preferably represents Cl or Br. Examples of E⁻ are ClO₄⁻, CF₃SO₃⁻, CH₃SO₃⁻, HSO₄⁻, BF₄⁻, B(phenyl)₄⁻, BARF (B(3,5-bis(trifluoromethyl)phenyl)₄⁻), PF₆⁻, SbCl₆⁻, AsF₆⁻ or SbF₆⁻.

In the process stage 1d), starting from α,β-unsaturated carboxylic acids of the formula VI, it is accordingly possible to use in particular metal complexes of the formulae IX and IXa, the ligands of which belong to the families of chiral ditertiary bisphosphines with a ferrocenyl backbone. Such ligands preferably correspond to the formula X or Xa,

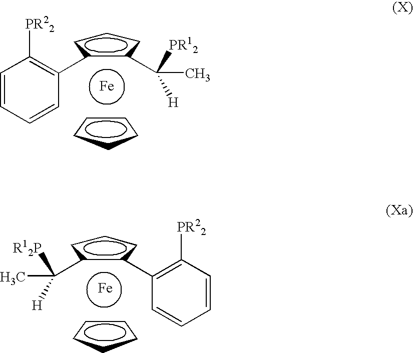

in which
R¹ can be C₃-C₈-cycloalkyl or aryl, and
R² can be C₃-C₈-cycloalkyl or aryl.

Examples of R¹ in the meaning of C₃-C₈-cycloalkyl are cyclohexyl and 2-norbornyl.

Examples of R¹ in the meaning of aryl are phenyl optionally substituted by 1 or 2 methyl, methoxy or trifluoromethyl groups.

An example of R² in the meaning of C₃-C₈-cycloalkyl is cyclohexyl.

Examples of R² in the meaning of aryl are phenyl optionally substituted by 1, 2 or 3 methyl, methoxy or trifluoromethyl groups.

Particular preference is given to ligands of the formulae X and Xa in which R¹ represents aryl and R² represents aryl and also to ligands of the formulae X and Xa in which R¹ represents aryl and R² represents C₃-C₈-cycloalkyl.

Preference is very particularly given to ligands of the formulae X and Xa in which

R¹ represents 3,5-bis(trifluoromethyl)phenyl and R² represents cyclohexyl or
R¹ represents 3,5-bis(trifluoromethyl)phenyl and R² represents phenyl or
R¹ represents 3,5-bis(trifluoromethyl)phenyl and R² represents 4-methoxy-3,5-dimethylphenyl.

Furthermore, it has been found that iridium metal complexes, the ligands of which belong to the families of chiral ditertiary bisphosphines with a ferrocenyl backbone, are surprisingly likewise suitable for the asymmetric hydrogenation of α,β-unsaturated carboxylic acids of the formula VI.

It is possible, with the abovementioned ferrocenyl ligand families in the metal complexes of the formulae XI and XIa described below, to obtain remarkably high enantiomeric purities, the use of iridium in place of rhodium representing a considerable advantage (for example, cost saving) for the preparation on an industrial scale.

[LM′YZ] (XI),

[LM′Y]⁺E⁻ (XIa),

in which
M′ represents iridium;
Y is two olefins or a diene;
Z represents Cl, Br or I;
E⁻ represents the anion of an oxo acid or complex acid; and
L is a chiral ligand from the group consisting of ditertiary bisphosphines.

When Y has the meaning olefin, C₂-C₁₂-olefins, preferably C₂-C₆-olefins and particularly preferably C₂-C₄-olefins may be concerned. Examples are propene, butene and in particular ethylene. The diene can comprise 5 to 12 and preferably 5 to 8 carbon atoms and open-chain, cyclic or polycyclic dienes may be concerned. The two olefin groups of the diene are preferably connected by one or two CH₂ groups. Examples are 1,3-pentadiene, cyclopentadiene, 1,5-hexadiene, 1,4-cyclohexadiene, 1,4- or 1,5-heptadiene, 1,4- or 1,5-cycloheptadiene, 1,4- or 1,5-octadiene, 1,4- or 1,5-cyclooctadiene and norbornadiene. Preferably, Y represents two ethylenes or 1,5-hexadiene, 1,5-cyclooctadiene or norbornadiene.

In formula IX, Z preferably represents Cl or Br. Examples of E⁻ are ClO₄⁻, CF₃SO₃⁻, CH₃SO₃⁻, HSO₄⁻, BF₄⁻, B(phenyl)₄⁻, BARF (B(3,5-bis(trifluoromethyl)phenyl)₄⁻), PF₆⁻, SbCl₆⁻, AsF₆— or SbF₆⁻.

In the process stage 1d), starting from α,β-unsaturated carboxylic acids of the formula VI, it is accordingly possible to use, for example, metal complexes of the formulae XI and XIa, the ligands of which belong to the families of chiral ditertiary bisphosphines with a ferrocenyl backbone.

Such ligands preferably correspond to the formula X or Xa,

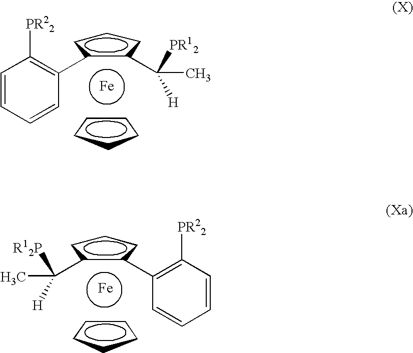

in which
R¹ and R² have the meanings given above.
Particular preference is given to ligands of the formulae X and Xa in which R¹ represents aryl and R² represents aryl and also to ligands of the formulae X and Xa in which R¹ represents aryl and R² represents C₃-C₈-cycloalkyl.
Very particular preference is given to ligands of the formulae X and Xa in which R¹ represents 3,5-bis(trifluoromethyl)phenyl and R² represents cyclohexyl;
   or
   to the formula XII or XIIa,

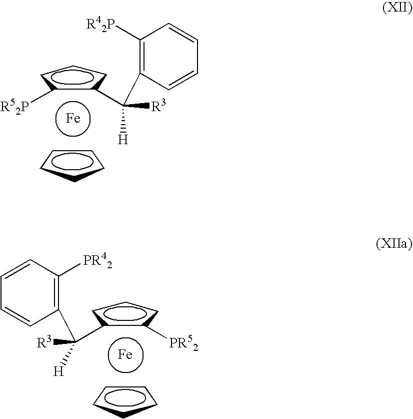

in which
R³ is dimethylamino,
R⁴ can be C₃-C₈-cycloalkyl or aryl,
R⁵ can be C₃-C₈-cycloalkyl or aryl.
An example of R⁴ and R⁵ in the meaning of C₃-C₈-cycloalkyl is cyclohexyl.
Examples of R⁴ and R⁵ in the meaning of aryl are phenyl optionally substituted by 1, 2 or 3 methyl, methoxy or trifluoromethyl groups.
Particular preference is given to ligands of the formulae XII and XIIa in which R⁴ and R⁵ represent phenyl;
   or
   to the formula XIII or XIIIa,

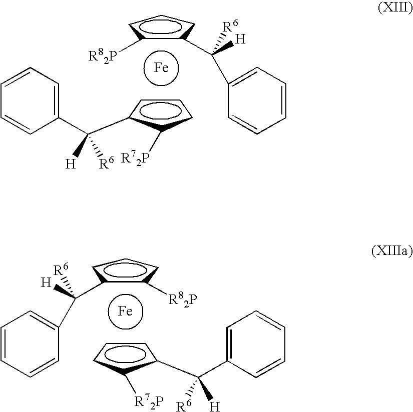

in which
R⁶ is dimethylamino,
R⁷ can be C₃-C₈-cycloalkyl or aryl,
R⁸ can be C₃-C₈-cycloalkyl or aryl.
An example of R⁷ and R⁸ in the meaning of C₃-C₈-cycloalkyl is cyclohexyl.
Examples of R⁷ and R⁸ in the meaning of aryl are phenyl optionally substituted by 1, 2 or 3 methyl, methoxy or trifluoromethyl groups.
Particular preference is given to ligands of the formulae XIII and XIIIa in which R⁷ and R⁸ represent aryl.
Preference is very particularly given to ligands of the formulae XIII and XIIIa in which R⁷ and R⁸ represent 4-methoxy-3,5-dimethylphenyl.

Asymmetric hydrogenations of the process stage 2e) or 3d) of α,β-unsaturated alcohols of the formula VIII can preferably be carried out using ruthenium, iridium and rhodium catalysts, as described, for example by M. Banziger and T. Troxler in Tetrahedron: Asymmetry, Vol. 14 (2003), pages 3469-3477, R. Gilbertson in Tetrahedron Letters, Vol. 44 (2003), pages 953-955, P. G. Andersson in the Journal of the American Chemical Society, Vol. 126 (2004), pages 14308-14309, A. Pfaltz in Organic Letters, Vol. 6 (2004), pages 2023-2026, and F. Spindler in Tetrahedron: Asymmetry, Vol. 15 (2004), pages 2299-2306.

Use is frequently made, as ligands for rhodium and ruthenium, of chiral ditertiary bisphosphines. Such chiral ditertiary bisphosphines are described, for example, by X. Zhang in Chemical Reviews, Vol. 103 (2003), pages 3029-3069.

Use is frequently made, as ligands for iridium, of chiral phosphine-oxazoline ligands or phosphinite-oxazoline ligands. Such chiral phosphine-oxazoline ligands or phosphinite-oxazoline ligands are described, for example, by A. Pfaltz in Advanced Synthesis and Catalysis, Vol. 345 (2003), pages 33-43.

It has now been surprisingly found that rhodium metal complexes, the ligands of which belong to the families of chiral ditertiary bisphosphines, are suitable in particular for the asymmetric hydrogenation of α,β-unsaturated alcohols of the formula VIII.

It is possible, with the abovementioned chiral ditertiary bisphosphines in the metal complexes of the formulae IX and IXa described below, to achieve high enantiomeric purities, which represents a considerable advantage (for example, cost saving) for the preparation on an industrial scale.

[LMYZ] (IX),

[LMY]⁺E⁻ (IXa),

in which
M, Y, Z, E⁻ and L have the meanings and preferences given above.

In the process stages 2e) and 3d), starting from α,β-unsaturated alcohols of the formula VIII, it is accordingly possible to use, for example, metal complexes of the formulae IX and IXa, the ligands of which belong to the families of chiral ditertiary bisphosphines with a ferrocenyl backbone. Such ligands preferably correspond to the formula XIV or XIVa,

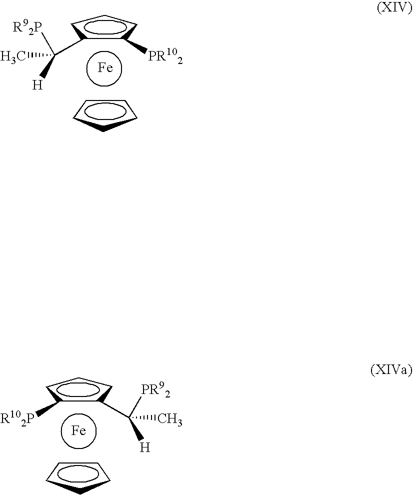

in which
R⁹ can be C₁-C₈-alkyl, C₃-C₈-cycloalkyl or aryl, and
R¹⁰ can be C₁-C₈-alkyl, C₃-C₈-cycloalkyl or aryl.
An example of R⁹ in the meaning of C₁-C₈-alkyl is t-butyl.
An example of R⁹ in the meaning of C₃-C₈-cycloalkyl is cyclohexyl.
Examples of R⁹ in the meaning of aryl are phenyl optionally substituted by 1 or 2 methyl groups.
Examples of R¹⁰ in the meaning of C₁-C₈-alkyl are ethyl and t-butyl.
An example of R¹⁰ in the meaning of C₃-C₈-cycloalkyl is cyclohexyl.
Examples of R¹⁰ in the meaning of aryl are phenyl optionally substituted by 1, 2 or 3 methyl, methoxy or trifluoromethyl groups, and also 2-furyl and 1-naphthyl.
Particular preference is given to ligands of the formulae XIV and XIVa in which R⁹ represents C₁-C₈-alkyl and R¹⁰ represents aryl and also to ligands of the formulae XIV and XIVa in which R⁹ represents aryl and R¹⁰ represents C₁-C₈-alkyl.
Preference is given very particularly to ligands of the formulae XIV and XIVa in which R⁹ represents phenyl and R¹⁰ represents t-butyl;
   or
   to the formula XV, XVI or XVII

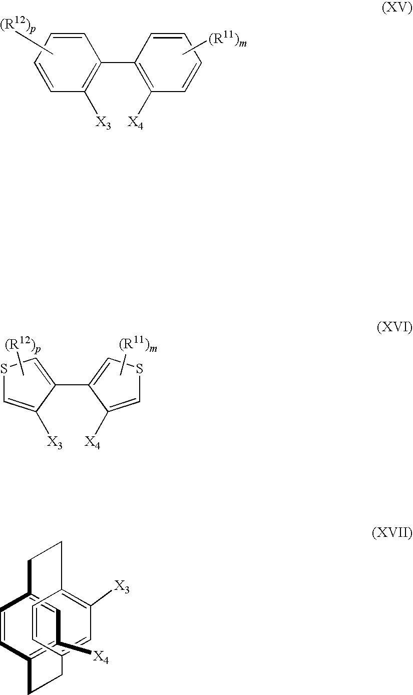

in which
m and p, in each case independently of one another, are 0 or an integer from 1 to 4 (XV) or are 0 or an integer 1 or 2 (XVI) and R¹¹ and R¹² represent hydrogen or identical or different substituents chosen from the group consisting of C₁-C₄-alkyl and C₁-C₄-alkoxy; and X₃ and X₄ represent, independently of one another, secondary phosphino.
With the ligands XV, the substituents are preferably bonded in the 6 position or the 6,6′ positions.
R¹¹ and R¹² can, as alkyl, preferably comprise 1 or 2 carbon atoms. Linear alkyl is preferred. Examples of R¹⁰ and R¹¹ in the meaning of alkyl are methyl, ethyl, n-propyl, isopropyl, n-butyl, isobutyl and t-butyl. Methyl and ethyl are preferred and methyl is particularly preferred.
R¹¹ and R¹² can, as alkoxy, preferably comprise 1 or 2 carbon atoms. Linear alkoxy is preferred. Examples of R¹¹ and R¹² in the meaning of alkoxy are methoxy, ethoxy, n-propoxy, isopropoxy, n-butoxy, isobutoxy and t-butoxy. Methoxy and ethoxy are preferred and methoxy is particularly preferred.
The X₃ and X₄ groups can be different or, preferably, identical and correspond to the formula PR¹³R¹⁴, in which R¹³ and R¹⁴ are identical or different and are branched C₃-C₈-alkyl, C₃-C₈-cycloalkyl, unsubstituted phenyl or phenyl substituted with one to three C₁-C₄-alkyl, C₁-C₄-alkoxy or —CF₃ groups.
Particular preference is given to ligands of the formula XV in which X₃ and X₄ represent a PR¹³R¹⁴ group in which R¹³ and R¹⁴ each represent cyclobutyl, cyclopentyl, cyclohexyl, phenyl or phenyl substituted with 1 or 2 methyl, methoxy or CF₃ groups.
Particular preference is likewise given to ligands of the formula XV, XVI and XVII in which X₃ and X₄ represent a PR¹³R¹⁴ group in which R¹³ and R¹⁴ represent phenyl.
Preference is very particularly given to ligands of the formula XV in which R¹¹ and R¹² represent methoxy and m and p represent 3.
Preference is likewise very particularly given to ligands of the formula XVI in which R¹¹ and R¹² represent methyl and m and p represent 2.

It has now likewise surprisingly been found that iridium metal complexes, the ligands of which belong to the families of chiral ditertiary bisphosphines, are suitable in particular for the asymmetric hydrogenation of α,β-unsaturated alcohols.

It is possible, with these ferrocenyl ligand families in the metal complexes of the formulae XI and XIa described below, to obtain high enantiomeric purities, which represents a considerable advantage (for example, cost saving) in comparison with the otherwise generally standard use of rhodium for the preparation on an industrial scale.

[LM′YZ] (XI),

[LM'Y]⁺E⁻ (XIa),

in which
M′, Y, Z, E⁻ and L have the meanings given above.

In the process stages 2e) and 3d), starting from α,β-unsaturated alcohols of the formula VIII, it is accordingly possible to use, for example, metal complexes of the formulae XI and XIa, the ligands of which belong to the families of chiral ditertiary bisphosphines with a ferrocenyl backbone. Such ligands preferably correspond to the formula XIV or XIVa,

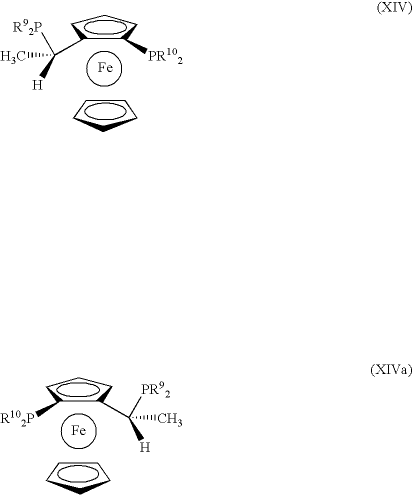

in which
R⁹ and R¹⁰ have the meanings and preferences given above;
or
to the formula XV

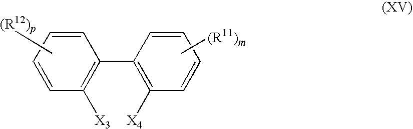

in which R¹¹ and R¹² and also X₃ and X₄ have the meanings and preferences given above;
ligands of the formula XV, in which R¹¹ and R¹² represent methoxy and m and p represent 1, with the R¹¹ and R¹² substituents in the 6,6′ position, likewise being very particularly preferred.

The metal complexes used as catalysts in the process stages 1d), 2e) and 3d) can be added as separately prepared isolated compounds or can also be formed in situ before the reaction and then mixed with the substrate to be hydrogenated. It can be advantageous, in the reaction using isolated metal complexes, to additionally add ligands or, in the in situ preparation, to use an excess of the ligands. The excess can, for example, be up to 10 mol and preferably from 0.001 to 5 mol, based on the metal compound used for the preparation.

The process stages 1d), 2e) and 3d) can be carried out at standard pressure or, preferably, under excess pressure. The pressure can, for example, be from 10⁵ to 2×10⁷ Pa (pascals).

Catalysts used for the hydrogenation in the process stages 1d), 2e) and 3d) are preferably used in amounts from 0.0001 to 10 mol %, particularly preferably from 0.001 to 10 mol % and especially preferably from 0.01 to 5 mol %, based on the compound to be hydrogenated.

The preparation of the catalysts as well as the process stages 1d), 2e) and 3d) and the other process stages can be carried out without or in the presence of an inert solvent, it being possible for a solvent or mixtures of solvents to be used. Suitable solvents are, for example, aliphatic, cycloaliphatic and aromatic hydrocarbons (pentane, hexane, petroleum ether, cyclohexane, methylcyclohexane, benzene, toluene, xylene), aliphatic halogenated hydrocarbons (methylene chloride, chloroform, dichloroethane and tetrachloroethane), nitriles (acetonitrile, propionitrile, benzonitrile), ethers (diethyl ether, dibutyl ether, t-butyl methyl ether, ethylene glycol dimethyl ether, ethylene glycol diethyl ether, diethylene glycol dimethyl ether, tetrahydrofuran, dioxane, diethylene glycol monomethyl or monoethyl ether), ketones (acetone, methyl isobutyl ketone), carboxylates and lactones (ethyl acetate, methyl acetate, valerolactone), N-substituted lactams (N-methylpyrrolidone), carboxamides (dimethylformamide), acyclic ureas (dimethylimidazoline), sulphoxides and sulphones (dimethyl sulphoxide, dimethyl sulphone, tetramethylene sulphoxide, tetramethylene sulphone), alcohols (methanol, ethanol, propanol, butanol, ethylene glycol monomethyl ether, ethylene glycol monoethyl ether, diethylene glycol monomethyl ether) and water. The solvents can be used alone or in a mixture of at least two solvents.

The reaction of the process stages 1d), 2e) and 3d) can be carried out in the presence of cocatalysts, for example quaternary ammonium halides (tetrabutylammonium iodide), and/or can be carried out in the presence of protic acids, for example inorganic acids.

The process stages 1e) and 2d) are preferably carried out at low temperatures, for example from −40° C. to 0° C., and advantageously in a solvent. Suitable solvents are, for example, ethers (tetrahydrofuran or dioxane). Metal hydrides in at least equimolar amounts are advisably used for the reduction, for example BH₃OS(CH₃)₂, LiAlH₄, NaBH₄+TiCl₄, NaBH₄+AlCl₃, NaBH₄+BF₃-Et₂O, LiAlH(OMe)₃ or AlH₃, and also alkylmetal hydrides, such as diisobutylaluminium hydride.

The process stage 3c) is preferably carried out at low temperatures, for example from 40° C. to 0° C., and advantageously in a solvent. Suitable solvents are, for example, hydrocarbons (pentane, cyclohexane, methylcyclohexane, benzene, toluene and xylene). Metal hydrides in at least equimolar amounts are advisably used for the reduction, for example NaBH₄, LiAlH₄ or AlH₃, and also alkylmetal hydrides, such as, for example diisobutylaluminium hydride and tributyltin hydride.

It is possible, with the regiospecific or regioselective and enantioselective process according to the invention, to prepare the intermediates for the preparation of the compound of the formula (B) over all process stages in high yields. The high overall yields make the process suitable for industrial use.

Another subject-matter of the invention is the compounds (intermediates)

of the formula V,

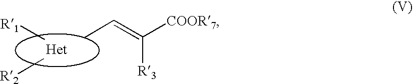

of the formula VI,

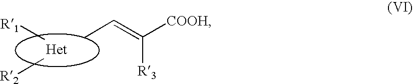

of the formula VII,

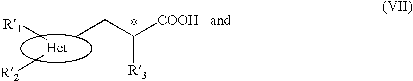

of the formula VIII,

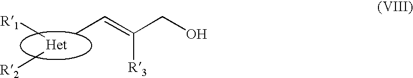

in which Het, R′₁, R′₂, R′₃ and R′₇ have the meanings given above and in which, for formula VII, the carbon atom to which the R′₃ radical is bonded exhibits either the (R) or (S) configuration, the (R) configuration being preferred.

Another subject-matter of the invention is the compound (intermediate) of the formula IV,

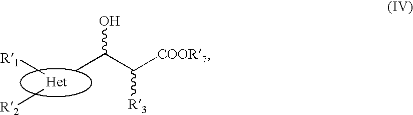

in which Het, R′₁, R′₂, R′₃ and R′₇ have the meanings given above.

The embodiments and preferences described above are valid for Het, R′₁, R′₂, R′₃ and R′₇.

The following examples explain the invention more fully.

HPLC gradient on Hypersil BDS C-18 (5 μm); column: 4×125 mm
(I) 90% water*/10% acetonitrile* to 0% water*/100% acetonitrile* in 5 minutes+2.5 minutes (1.5 ml/min)
(II) 95% water*/5% acetonitrile* to 0% water*/100% acetonitrile* in 40 minutes (0.8 ml/min)
   HPLC gradient on Synergi Polar-RP 80A(Phenomenex) (4 μm); column: 4.60×100 mm
(III) 90% water*/10% acetonitrile* to 0% water*/100% acetonitrile* in 5 minutes+2.5 minutes (1.5 m/min)
(IV) 95% water*/5% acetonitrile* to 0% water*/100% acetonitrile* in 40 minutes (0.8 ml/min)
   * comprises 0.1% of trifluoroacetic acid

### EXAMPLE A

### Process for the Preparation of (R)-2-[1-(3-methoxypropyl)-3-methyl-1H-indazol-6-ylmethyl]-3-methylbutan-1-ol (A6)

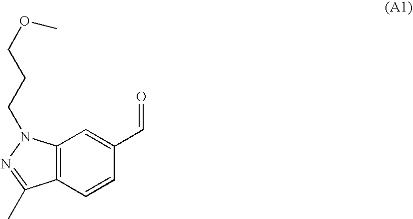

### EXAMPLE A1

### Preparation of 1-(3-methoxypropyl)-3-methyl-1H-indazole-6-carbaldehyde

A solution of 10.0 g of 6-bromo-1-(3-methoxypropyl)-3-methyl-1H-indazole (WO 2005/090305 A1) in 70 ml of tetrahydrofuran is cooled to −78° C. and treated with 3.76 ml of N-methylmorpholine. Cooling is again carried out to −78° C. and 23 ml of n-butyllithium (1.6M in hexane) are added dropwise so that the internal temperature does not climb above −70° C. Stirring is carried out at −70° C. for a further 2 minutes. Subsequently, 5.18 ml of N,N-dimethylformamide are added dropwise so that the internal temperature does not climb above −70° C. Stirring is carried out at −70° C. for a further 5 minutes. 70 ml of a 1 M aqueous ammonium chloride solution are added to the reaction mixture and the latter is heated to ambient temperature. The reaction mixture is diluted with 50 ml of water and subsequently extracted with tert-butyl methyl ether (2×100 ml). The organic phases are washed with aqueous saline solution (1×100 ml). The combined organic phases are dried over sodium sulphate, filtered and evaporated on a rotary evaporator. The crude title compound A1 is obtained from the residue as a yellow oil. Content (NMR): 90% (comprises 10% of 1-(3-methoxypropyl)-3-methyl-1H-indazole). (7.80 g, 90.4%). Rf=0.27 (acetic ester/heptane 1:1); Rt=3.67 (Gradient I).

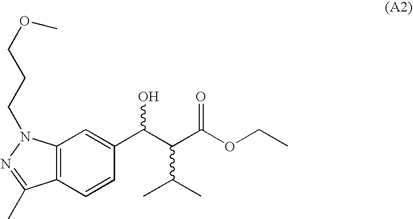

### EXAMPLE A2

### Preparation of ethyl 2-hydroxy[1-(3-methoxypropyl)-3-methyl-1H-indazol-6-yl]methyl)-3-methylbutyrate

A solution of 2.628 ml of diisopropylamine and 20 ml of tetrahydrofuran is cooled to −20° C. and 11.508 ml of n-butyllithium (1.6M in hexane) are added dropwise over 7 minutes. Stirring is carried out at −20° C. for a further 10 minutes. Subsequently, a solution of 2.62 ml of ethyl isovalerate in 15 ml of tetrahydrofuran is added dropwise over 10 minutes at −20° C. After a further 5 minutes a solution of 3.60 g of 1-(3-methoxypropyl)-3-methyl-1H-indazole-6-carbaldehyde (A1) in 15 ml of tetrahydrofuran is added dropwise and stirring is carried out at −20° C. for a further 30 minutes. 30 ml of saturated aqueous ammonium chloride solution are then added dropwise and extraction is subsequently carried out with tert-butyl methyl ether (2×100 ml). The organic phases are successively washed with 0.5N hydrochloric acid (1×100 ml) and aqueous saline solution (1×50 ml). The combined organic phases are dried over sodium sulphate, filtered and evaporated on a rotary evaporator. The crude title compound A2 is obtained from the residue as a white solid (4.98 g, 89.6%, syn:anti=67:33). Rf=0.08 (acetic ester/heptane 1:2); Rt=15.96, 16.75 (Gradient II).

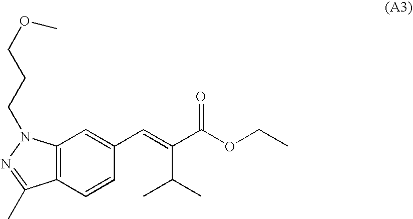

### EXAMPLE A3

### Preparation of ethyl 2-[1-[1-(3-methoxypropyl)-3-methyl-1H-indazol-6-yl]meth-(E)-ylidene]-3-methylbutyrate

A solution of 2.80 g of ethyl 2-hydroxy[1-(3-methoxypropyl)-3-methyl-1H-indazol-6-yl]methyl)-3-methylbutyrate (A2) and 47 mg of 4-dimethylaminopyridine in 20 ml of tetrahydrofuran is cooled to 0° C. 0.79 ml of acetic anhydride is added dropwise over 2 minutes and the reaction mixture is stirred at 0° C. for 1 hour. A solution of 2.63 g of potassium tert-butoxide in 20 ml of tetrahydrofuran is then added dropwise at 0° C. over 1 hour and stirring is subsequently carried out at 0° C. for 1 hour. The reaction mixture is poured onto 100 ml of ice-cold water and extracted with tertbutyl methyl ether (2×80 ml). The organic phases are successively washed with 80 ml of water and 80 ml of aqueous saline solution, dried over sodium sulphate, filtered and evaporated on a rotary evaporator. The pure title compound A3 is obtained from the residue by means of flash chromatography (SiO₂ 60F, acetic ester/hexane 1:6) as a light yellowish oil (1.83 g, 70%). Rf=0.28 (acetic ester/heptane 1:2); Rt=22.42 (Gradient II).

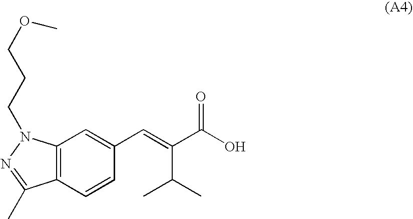

### EXAMPLE A4

### Preparation of 2-[1-[1-(3-methoxypropyl)-3-methyl-1H-indazol-6-yl]meth-(E)-ylidene]-3-methylbutyric Acid

A solution of 2.80 g of ethyl 2-{hydroxy[1-(3-methoxypropyl)-3-methyl-1H-indazol-6-yl]methyl}-3-methylbutyrate (A2) and 47 mg of 4-dimethylaminopyridine in 20 ml of tetrahydrofuran is cooled to 0° C. 0.79 ml of acetic anhydride is added dropwise over 2 minutes and the reaction mixture is stirred at 0° C. for 1 hour. A solution of 2.63 g of potassium tert-butoxide in 20 ml of tetrahydrofuran is then added dropwise at 0° C. over 1 hour and stirring is subsequently carried out at 0° C. for 1 hour. 10 ml of ice-cold water are added dropwise to the reaction mixture over 1 minute and the tetrahydrofuran is evaporated on a rotary evaporator. The aqueous emulsion is treated with 28 ml of ethanol and 3.8 ml of 2M aqueous potassium hydroxide solution and heated at reflux for 13.5 hours. The ethanol is evaporated from the reaction mixture on a rotary evaporator (35° C.). The resulting aqueous solution is washed with tert-butyl methyl ether (2×15 ml). The aqueous phase is acidified with 10 ml of 2M aqueous hydrochloric acid solution and extracted with tert-butyl methyl ether (2×30 ml). The organic phases are successively washed with 15 ml of water and 15 ml of aqueous saline solution, dried over sodium sulphate, filtered and evaporated on a rotary evaporator. The pure title compound A4 is obtained as white crystals from the residue by means of crystallization from a hot acetic ester/heptane mixture (1.44 g, 60.1%). Rf=0.27 (acetic ester/heptane 3:1); Rt=16.41 (Gradient II).

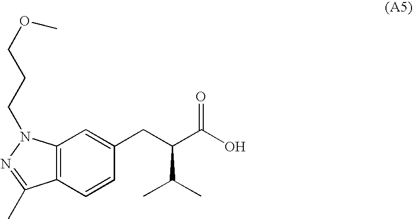

### EXAMPLE A5

### (R)-2-[1-(3-Methoxypropyl)-3-methyl-1H-indazol-6-ylmethyl]-3-methylbutyric Acid

The title compound can be obtained in the form of white crystals by catalytic asymmetric hydrogenation of 2-[1-[1-(3-methoxypropyl)-3-methyl-1H-indazol-6-yl]meth-(E)-ylidene]-3-methylbutyric acid (A4) and purification of the residue by means of flash chromatography (SiO₂ 60F, acetic ester). Rf=0.32 (acetic ester/heptane 2:1); Rt=4.03 (Gradient I).

The asymmetric hydrogenations of 2-[1-[1-(3-methoxypropyl)-3-methyl-1H-indazol-6-yl]meth-(E)-ylidene]-3-methylbutyric acid (A4) are carried out in a fully automated high throughput screening unit developed by Symyx.

The reaction mixture is investigated for conversion and enantiomeric excess using the HPLC method mentioned below. For this, 80 μl of the reaction solution are dissolved in 1000 μl of ethanol. The following results are obtained:

| | | | | | | ee % |
|---|---|---|---|---|---|---|
| | | | | | | (abso- |
| | | Sub- | | | | lute |
| | | strate/ | | | Con- | con- |
| | Metal | catalyst | | Addi- | ver- | figur- |
| Ligand | precursor | ratio | Solvent | tive | sion | ation) |
| 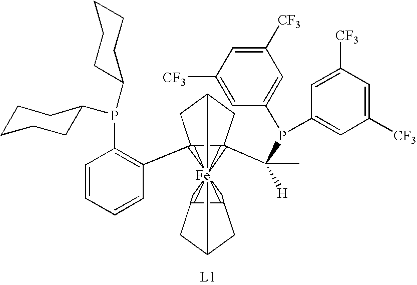 | [Rh(NBD)₂]BF₄[Rh(NBD)₂]BF₄[Rh(NBD)Cl]₂[Rh(NBD)(OOCF₃)]₂ | 25252525 | THFMeOHDCEDCE | DABCO | >90>90>90>90 | 94.84(R)93.10(R)95.12(R)93.11(R) |
| 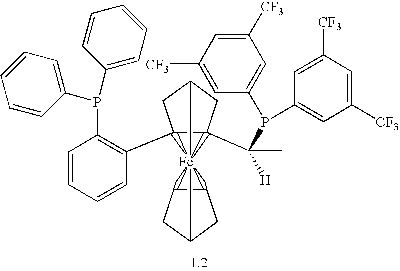 | [Rh(NBD)₂]BF₄[Rh(NBD)(OOCF₃)]₂[Rh(NBD)₂]BF₄[Rh(NBD)₂]BF₄[Rh(NBD)₂]BF₄ | 25251002525 | MeOHMeOHMeOHMeOHEthanol | DABCO | >90>90>90>90>90>90 | 95.07(R)94.75(R)94.53(R)94.52(R)94.23(R) |
| 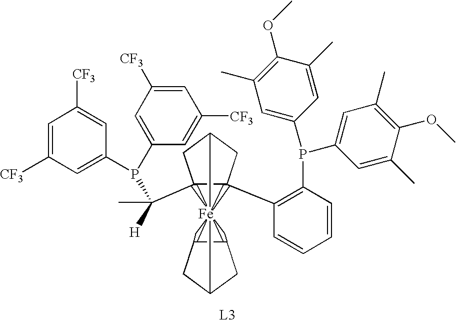 | [Rh(NBD)₂]BF₄ | 25 | MeOH | | >90 | 94.39(S)* |
| 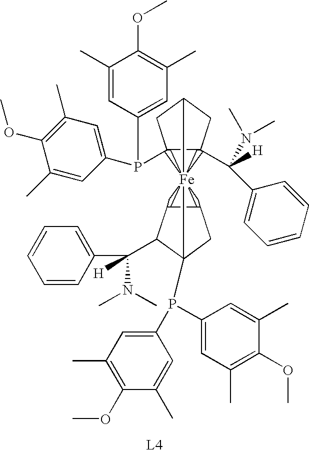 | [Ir(COD)₂]BF₄[Ir(COD)Cl]₂[Ir(COD)₂]BARF | 252525 | DCEDCEMeOH | DABCOTEA | >90>90>90 | 74.91(R)80.42(R)79.19(R) |
| 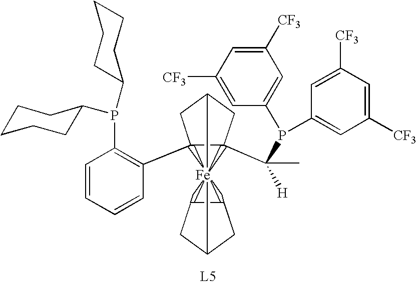 | [Ir(COD)₂]BF₄ | 25 | DCE | | >90 | 74.60(R) |
| 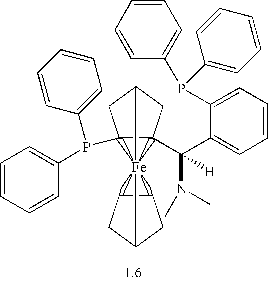 | [Ir(COD)₂]BF₄ | 25 | DCE | | >90 | 76.26(S)* |

Conditions: 41.66 μmol of 2-[1-[1-(3-methoxypropyl)-3-methyl-1H-indazol-6-yl]meth-(E)-ylidene]-3-methylbutyric acid (A4); 500 μl of solvent;
1.2 equivalents of ligand per metal; p(H₂): 20 bar;
T: ambient temperature; reaction time: 16 hours
*Under otherwise identical conditions, the product with the (R) configuration is obtained with the enantiomenc ligand.

| | |
|---|---|
| HPLC conditions: | |
| Instrument | SFC Berger Instruments |
| Column | CHIRALPAK-AD (250 mm * 0.46 cm) |
| Modifier | Ethanol |
| Outlet pressure | 100 bar |
| Gradient | 15% EtOH 8′, 60%/min 40%, 2′ 40%, 60%/min 15%, 15% 6′, Total 17′ |
| Flow rate | 1.5 ml/min. |
| Detection | UV (210 nm) |
| Temperature | 40° C. |
| Sample concentration | 2 mg product in 1.0 ml MeOH |
| Injection volume | 5.0 μl loop |
| Run time | 12 min. |
| Retention times: | |
| (S)-(A5) | 4.7 min |
| (R)-(A5) | 5.8 min |
| (A4) | 8.5 min |
| 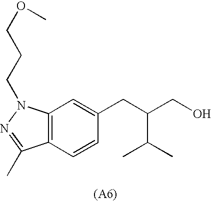 | |

### EXAMPLE A6

### (R)-2-[1-(3-Methoxypropyl)-3-methyl-1H-indazol-6-ylmethyl]-3-methylbutan-1-ol

### a) Preparation Starting from (R)-2-[1-(3-methoxypropyl)-3-methyl-1H-indazol-6-ylmethyl]-3-methylbutyric Acid (A5)

A solution of 8.55 g of (R)-2-[1-(3-methoxypropyl)-3-methyl-1H-indazol-6-ylmethyl]-3-methylbutyric acid (A5) in 85 ml of tetrahydrofuran is cooled to 0° C. and treated with 81.4 ml of borane-tetrahydrofuran complex (1M in tetrahydrofuran). The reaction mixture is stirred at ambient temperature for 17 hours. The reaction mixture is cooled down to 0° C. and subsequently treated slowly with 100 ml of methanol. The mixture is evaporated on a rotary evaporator and dried under high vacuum. The pure title compound A6 is obtained as a colourless oil from the residue by means of flash chromatography (SiO₂ 60F, acetic ester/hexane 2:1) (8.05 g, 98%). Rf=0.28 (acetic ester/heptane 2:1); Rt=4.13 (Gradient I).

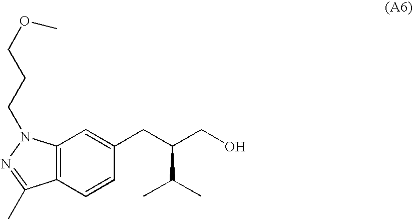

### EXAMPLE A6

### (R)-2-[1-(3-Methoxypropyl)-3-methyl-1H-indazol-6-ylmethyl]-3-methylbutan-1-ol

### b) Preparation Starting from 2-[1-[1-(3-methoxypropyl)-3-methyl-1H-indazol-6-yl]meth-(E)-ylidene]-3-methylbutan-1-ol (A7)

The title compound can be obtained as a light pink oil by catalytic asymmetric hydrogenation of 2-[1-[1-(3-methoxypropyl)-3-methyl-1H-indazol-6-yl]meth-(E)-ylidene]-3-methylbutan-1-ol (A7) and purification of the residue by means of flash chromatography (SiO₂ 60F, acetic ester). Rf=0.32 (acetic ester/heptane 2:1); Rt=4.13 (Gradient I).

The asymmetric hydrogenations of 2-[1-[1-(3-methoxypropyl)-3-methyl-1H-indazol-6-yl]meth-(E)-ylidene]-3-methylbutan-1-ol (A7) are carried out in a fully automatic high throughput screening unit developed by Symyx.

Conditions: 41.66 μmol of substrate, 500 μl of solvent, 1.2 equivalents of ligand per metal. The reaction mixture is investigated for conversion and enantiomeric excess using the HPLC method mentioned below. For this, 80 μl of the reaction solution are dissolved in 1000 μl of ethanol. The following results are obtained:

| | | | | | ee % |
|---|---|---|---|---|---|
| | | | | | (abso- |
| | | Sub- | | | lute |
| | | strate/ | | Con- | con- |
| | Metal | catalyst | | ver- | figur- |
| Ligand | precursor | ratio | Solvent | sion | ation) |
| 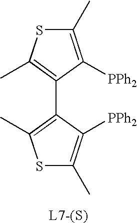 | [Rh(NBD)Cl]₂[Rh(NBD)Cl]₂ | 50200 | TolueneToluene | 100100 | 97.9 (S)*97.9 (S)* |
| 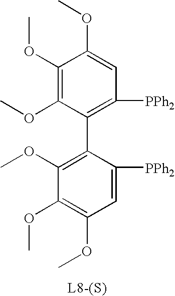 | [Rh(NBD)Cl]₂[Rh(NBD)Cl]₂ | 50200 | TolueneToluene | 100100 | 95.0 (S)*93.9 (S)* |
| 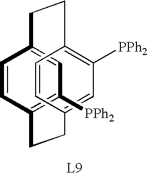 | [Rh(NBD)Cl]₂ | 50 | Toluene | 100 | 93.3 (S)* |
| 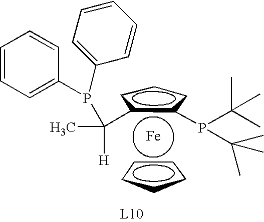 | [Rh(NBD)Cl]₂[Rh(NBD)Cl]₂ | 50200 | TolueneToluene | 100100 | 95.7 (S)*93.1 (S)* |
| 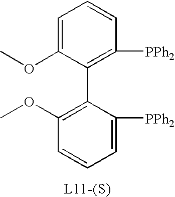 | [Ir(COD)Cl]₂ | 50 | DCE | >90 | 80.42 (R) |
| 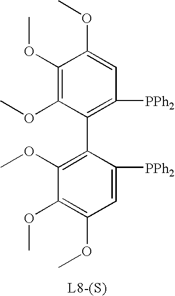 | [Ir(COD)Cl] | 50 | MeOH | >90 | 79.19 (S)* |
| 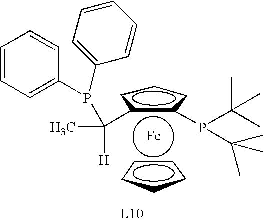 | [Ir(COD)Cl] | 50 | DCE | >90 | 74.60 (S)* |

Conditions: 41.66 μmol of 2-[1-[1-(3-methoxypropyl)-3-methyl-1H-indazol-6-yl]meth-(E)-
ylidene]-3-methylbutan-l-ol (A7); 500 μl of solvent; 1.2 equivalents of ligand per metal;
p(H₂): 80 bar; T: 40° C.; reaction time: 14 hours
*Under otherwise identical conditions, the product with the (R) configuration is obtained
with the enantiomeric ligand.

| | |
|---|---|
| HPLC conditions: | |
| Instrument | SEC Berger Instruments |
| Column | CHIRALPAK-AD (250 mm * 0.46 cm) |
| Modifier | Ethanol |
| Outlet pressure | 100 bar |
| Gradient | 15% EtOH 8′, 60%/min 40%, 2′ 40%, 60%/min 15%, 15% 6′, |
| | Total 17′ |
| Flow rate | 1.5 ml/min. |
| Detection | UV (210 nm) |
| Temperature | 40° C. |
| Sample concentration | 2 mg product in 1.0 ml MeOH |
| Injection volume | 5.0 μl loop |
| Run time | 12 min. |
| Retention time: | |
| (S)-(A6) | 6.1 min |
| (R)-(A6) | 7.5 min |
| (A7) | 8.2 min |

Representative description of the implementation of the reaction on a larger scale:

A solution of 1.65 mmol of 2-[1-[1-(3-methoxypropyl)-3-methyl-1H-indazol-6-yl]meth-(E)-ylidene]-3-methylbutan-1-ol (A7) in 4 ml of degassed dry solvent is prepared in a Schlenk tube and stirred at ambient temperature for 10 minutes. A solution of the appropriate amount of the ligand and of the metal precursor (1.05 equivalents of ligand per metal) in 4 ml of degassed dry solvent is prepared in a second Schlenk tube under an argon atmosphere and the mixture is stirred at ambient temperature for 10 minutes. The two solutions are transferred via a hollow needle into a 50 ml autoclave made of special stainless steel which has been placed under an argon atmosphere beforehand. The autoclave is closed and flushed with argon (4 times a pressure of 10-12 bar is imposed on each occasion and is again relaxed on each occasion to 1 bar). Subsequently, the argon is replaced by hydrogen and flushing is carried out with hydrogen (4 times a pressure of 10-12 bar is imposed on each occasion and is again relaxed on each occasion to 1 bar). The autoclave is subsequently placed under a pressure of 80 bar with hydrogen and heated to 40° C. After 20 hours, cooling is carried out to ambient temperature and the pressure is removed.

The reaction mixture is investigated for conversion and enantiomeric excess using the HPLC method mentioned above. For this, 80 μl of the reaction solution are dissolved in 1000 μl of ethanol. The following results are obtained:

| | | | | | ee % |
|---|---|---|---|---|---|
| | | | | | (abso- |
| | | Sub- | | | lute |
| | | strate/ | | Con- | con- |
| | Metal | catalyst | | ver- | figur- |
| Ligand | precursor | ratio | Solvent | sion | ation) |
| 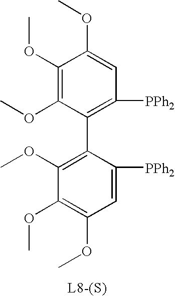 | [Rh(NBD)Cl]₂ | 100 | Toluene | 100 | 93.4 (S)* |

Conditions: 3.306 mmol of 2-[1-[1-(3-methoxypropyl)-3-methyl-1H-indazol-6-yl]meth-(E)-ylidene]-3-methylbutan-1-ol (A7); 10 ml of solvent; p(H₂): 80 bar; T: 40° C.; reaction time: 17 hours
*Under otherwise identical conditions, the product with the (R) configuration is obtained with the enantiomeiic ligand.

| | | | | | ee % |
|---|---|---|---|---|---|
| | | | | | (abso- |
| | | Sub- | | | lute |
| | | strate/ | | Con- | con- |
| | Metal | catalyst | | ver- | figur- |
| Ligand | precursor | ratio | Solvent | sion | ation) |
| 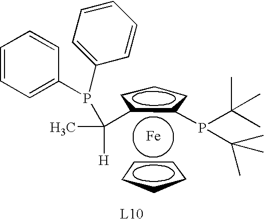 | [Rh(NBD)Cl]₂ | 100 | Toluene | 100 | 95.6 (S)* |

Conditions: 1.65 mmol of 2-[1-[1-(3-methoxypropyl)-3-methyl-1H-indazol-6-yl]meth-(E)-ylidene]-3-methylbutan-1-ol (A7); 10 ml of solvent; p(H₂): 80 bar; T: 40° C.; reaction time: 17 hours
*Under otherwise identical conditions, the product with the (R) configuration is obtained with the enantiomeric ligand.

Alternatively, the title compound (A6) can be obtained by reduction of 2-[1-[1-(3-methoxypropyl)-3-methyl-1H-indazol-6-yl]meth-(E)-ylidene]-3-methylbutyric acid (A4) to give 2-[1-[1-(3-methoxypropyl)-3-methyl-1H-indazol-6-yl]meth-(E)-ylidene]-3-methylbutan-1-ol (A7) and subsequent catalytic asymmetric hydrogenation.

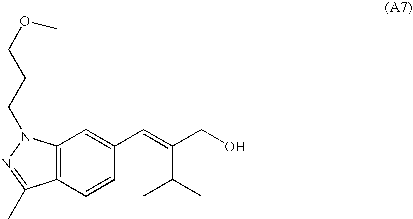

### EXAMPLE A7

### 2-[1-[1-(3-Methoxypropyl)-3-methyl-1H-indazol-6-yl]meth-(E)-ylidene]-3-methylbutan-1-ol

### a) Preparation Starting from 2-[1-[1-(3-methoxypropyl)-3-methyl-1H-indazol-6-yl]meth-(E)-ylidene]-3-methylbutyric Acid (A4)

A solution of 470 mg of (2-[1-[1-(3-methoxypropyl)-3-methyl-1H-indazol-6-yl]meth-(E)-ylidene]-3-methylbutyric acid (A4) in 2 ml of tetrahydrofuran is cooled to 0° C. and treated with a solution of 56.4 mg of lithium aluminium hydride in 2.5 ml of tetrahydrofuran. The reaction mixture is stirred at ambient temperature for 19 hours. Subsequently, 50.0 mg of solid lithium aluminium hydride are added at ambient temperature and the reaction mixture is stirred at ambient temperature for 2 hours. The reaction mixture is slowly treated with 3 ml of glacial acetic acid. The mixture is washed with potassium sodium tartrate solution, the aqueous phases are extracted with tert-butyl methyl ether and the combined organic phases are dried over sodium sulphate, filtered and evaporated on a rotary evaporator, and the residue is dried under high vacuum. The pure title compound A7 is obtained as a yellow solid from the residue by means of flash chromatography (SiO₂ 60F, acetic ester/hexane 2:1) (252.7 mg, 56%). Rf=0.29 (acetic ester/heptane 2:1); Rt=3.96 (Gradient I).

Alternatively, the title compound (A6) can be obtained by catalytic reduction of ethyl 2-[1-[1-(3-methoxypropyl)-3-methyl-1H-indazol-6-yl]meth-(E)-ylidene]-3-methylbutyrate (A3) to give 2-[1-[1-(3-methoxypropyl)-3-methyl-1H-indazol-6-yl]meth-(E)-ylidene]-3-methylbutan-1-ol (A7) and subsequent catalytic asymmetric hydrogenation.

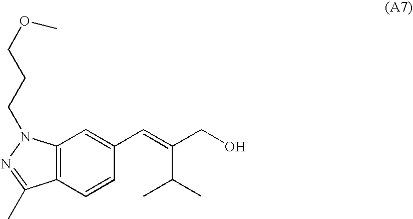

### EXAMPLE A7

### 2-[1-[1-(3-Methoxypropyl)-3-methyl-1H-indazol-6-yl]meth-(E)-ylidene]-3-methylbutan-1-ol

### b) Preparation Starting from ethyl 2-[1-[1-(3-methoxypropyl)-3-methyl-1H-indazol-6-yl]meth-(E)-ylidene]-3-methylbutyrate (A3)

A solution of 19.68 g of ethyl 2-[1-[1-(3-methoxypropyl)-3-methyl-1H-indazol-6-yl]meth-(E)-ylidene]-3-methylbutyrate (A3) in 433 ml of toluene is cooled to −20° C. and treated with a solution of 115.2 ml of diisobutylaluminium hydride (1.7M in toluene), the temperature being maintained at −20° C. The reaction mixture is subsequently heated to ambient temperature and stirred at ambient temperature for 1 hour. Subsequently, the reaction mixture is slowly treated with 1 l of 1 M HCl, the temperature being maintained at less than 30° C. The phases are separated and the aqueous phase is extracted with diethyl ether (1×1l, 2×300 ml). The combined organic phases are successively washed with 1 l each of water, saturated aqueous sodium carbonate solution and aqueous saline solution, dried over sodium sulphate, filtered and evaporated on a rotary evaporator, and the residue is dried under high vacuum. The pure title compound A7 is obtained as a yellow oil from the residue by means of flash chromatography (SiO₂ 60F, dichloromethane/methanol/conc. ammonia 200:5:1) (14.24 g, 82%). Rf=0.29 (dichloromethane/methanol/conc. ammonia 200:5:1); Rt=3.96 (Gradient I).

The following compound can be prepared analogously according to the process described in Example A:

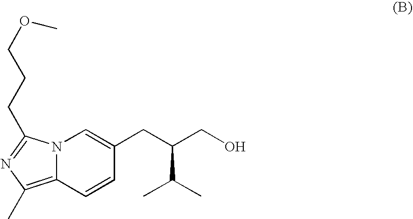

B) (R)-2-[3-(3-Methoxy-propyl)-1-methyl-imidazo[1,5-a]pyridin-6-ylmethyl]-3-methyl-butan-1-ol

1-10. (canceled)

11. A process for the preparation of a compound of the formula I 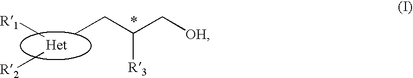
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, and R′₃ represents isopropyl, and in which the carbon atom to which the R′₃ radical is bonded exhibits either the (R) or (S) configuration, the (R) configuration being preferred, characterized in that
a) a compound of the formula II 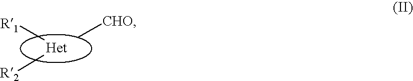
in which Het, R′₁ and R′₂ have the meanings given above, is reacted with a compound of the formula III, 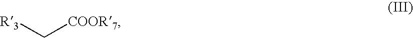
in which R′₃ has the meaning given above, to give a diastereomeric mixture of the formula IV, 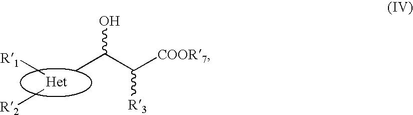
in which R′₇ is C₁-C₁₂-alkyl, C₃-C₈-cycloalkyl, phenyl or benzyl,
b) the OH group of the diastereomeric mixture of the formula IV is converted to a leaving group and the leaving group is eliminated in the presence of a strong base to give an acrylate of the formula V, 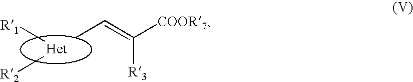
subsequently either according to process variant 1:
1c) the acrylate of the formula V is converted by saponification to give a compound of the formula VI, 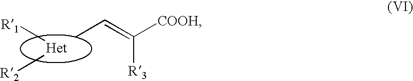
1d) the acid of the formula VI is hydrogenated in the presence of hydrogen and catalytic amounts of a metal complex as an asymmetric hydrogenation catalyst, which comprises metals from the group consisting of ruthenium, rhodium and iridium to which chiral bidentate ligands are bonded, to give a compound of the formula VII, 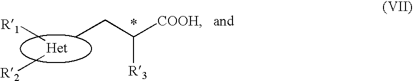
1e) the acid of the formula VII is reduced to give a compound of the formula I;
or according to process variant 2:
2c) the acrylate of the formula V is converted by saponification to give a compound of the formula VI, 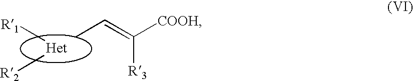
2d) the acid of the formula VI is reduced to give a compound of the formula VIII 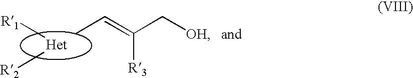
2e) the alcohol of the formula VIII is hydrogenated in the presence of hydrogen and catalytic amounts of a metal complex as an asymmetric hydrogenation catalyst, which comprises metals from the group consisting of ruthenium, rhodium and iridium to which chiral bidentate ligands are bonded, to give a compound of the formula I;
or according to process variant 3:
3c) the acrylate of the formula V is reduced to give a compound of the formula VIII 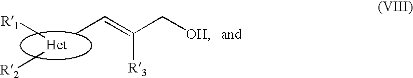
3d) the alcohol of the formula VIII is hydrogenated in the presence of hydrogen and catalytic amounts of a metal complex as an asymmetric hydrogenation catalyst, which comprises metals from the group consisting of ruthenium, rhodium and iridium to which chiral bidentate ligands are bonded, to give a compound of the formula I.

12. A process according to claim 11, wherein R′₁ represents methoxy- or ethoxy-C₁-C₄-alkyl, and R′₂ represents methyl, ethyl, methoxy or ethoxy.

13. A process according to claim 11 or 12, wherein Het represents either 1H-indol-6-yl or 1H-indazol-6-yl.

14. A process according to claim 11 or 12, wherein Het substituted with R′₁ and R′₂ represents either 1-(3-methoxypropyl)-3-methyl-1H-indol-6-yl or 1-(3-methoxypropyl)-3-methyl-1H-indazol-6-yl and R′₃ representing isopropyl.

15. A compound of the formula V, 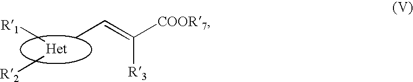
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, R′₃ represents isopropyl and R′₇ is C₁-C₁₂-alkyl, C₃-C₈-cycloalkyl, phenyl or benzyl.

16. A compound of the formula VI, 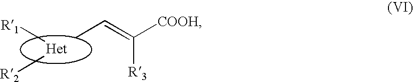
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, and R′₃ represents isopropyl.

17. A compound of the formula VII, 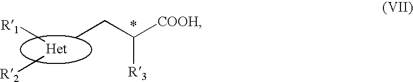
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, and R′₃ represents isopropyl and in which the carbon atom to which the R′₃ radical is bonded exhibits either the (R) or (S) configuration, the (R) configuration being preferred.

18. A compound of the formula VIII, 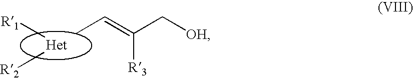
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, and R′₃ represents isopropyl.

19. A compound of the formula IV, 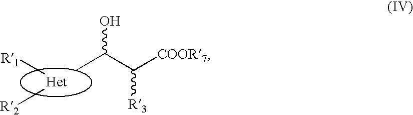
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, R′₃ represents isopropyl and R′₇ is C₁-C₁₂-alkyl, C₃-C₈-cycloalkyl, phenyl or benzyl.

20. A process according to claim 11, whereby a compound of formula (I), prepared according to claim 11, is, by halogenation, transferred to a compound formula B 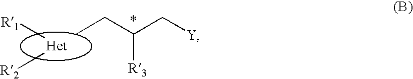
in which Het represents an unsaturated bicyclic heterocyclic-radical with one or two nitrogen atoms as heteroatoms, joined via a carbon atom to the residual molecule, the ring not directly bonded to the residual molecule being substituted by R′₁ and R′₂, R′₁ and R′₂ represent, independently of one another, H, C₁-C₈-alkyl, halogen, polyhalo-C₁-C₈-alkoxy, polyhalo-C₁-C₈-alkyl, C₁-C₈-alkoxy, C₁-C₈-alkoxy-C₁-C₈-alkyl or C₁-C₈-alkoxy-C₁-C₈-alkoxy, R′₁ and R′₂ not simultaneously representing H, R′₃ represents isopropyl, and in which the carbon atom to which the R′₃ radical is bonded exhibits either the (R) or (S) configuration, the (R) configuration being preferred, and Y represents Cl, Br or I,
said compound of formula B is further reacted, in the presence of an alkali metal or alkaline earth metal, with a compound of the formula C 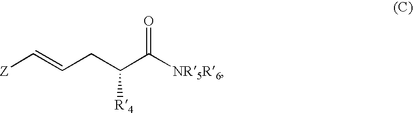
wherein R′₄ is C₁-C₈-alkyl, R′₅ represents C₁-C₈-alkyl or C₁-C₈-alkoxy, R′₆ represents C₁-C₈-alkyl or R′₅ and R′₆ together are tetramethylene, pentamethylene, 3-oxa-1,5-pentylene or —CH₂CH₂O—C(O)— optionally substituted by C₁-C₄-alkyl, phenyl or benzyl,
and Z represents Cl, Br or I,
to give a compound of the formula A 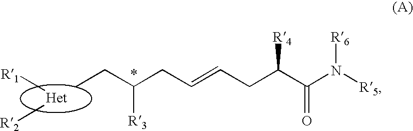
in which Het and R′₁ to R′₆ have the meanings given above, and in which the carbon atom to which the R′₃ radical is bonded exhibits either the (R) or (S) configuration, the (R) configuration being preferred.
